(19) United States
(12) Patent Application Publication
(10) Pub. No.: US 2009/0318754 A1
(43) Pub. Date: Dec. 24, 2009

(54) PENIS EXTENSION SYSTEM INTEGRATED INTO AN ITEM OF CLOTHING

(76) Inventor: Jürgen Ettmer, Hannover (DE)

Correspondence Address: REMENICK PLLC, 1025 THOMAS JEFFERSON STREET, NW, WASHINGTON, DC, 20007, US

(21) Appl. No.: 12/519,076

(22) PCT Filed: Nov. 16, 2007
(86) PCT No.: PCT/DE07/02115

§ 371 Date: Aug. 20, 2009

(30) Foreign Application Priority Data
Nov. 16, 2006 (DE) 10 2006 054 642.3
Mar. 9, 2007 (DE) 10 2007 012 319.3
Apr. 23, 2007 (DE) 10 2007 020 236.0

(51) Int. Cl. A61F 5/00; A41B 9/02

(52) U.S. Cl. 600/38; 2/403

(57) ABSTRACT

The invention relates to a penis extension system that can be integrated into the clothing of a user and worn as the user moves around, with the aim of increasing the size of the penis and the glans. Said system comprises a cradle which is used to receive the glans and can be fixed to the shaft of the penis. One end of a traction element (2) is fixed to the cradle and the other end of the traction element can be detachably fixed to a first point on the clothing of a user by means of a first fixing device (7). The first point on the clothing depends on the tractive force and on the direction of the tractive force to be transferred to the penis by means of the tractive element. Said system also comprises an adjustment arranged on the base of the penis of the user, said abutment being in the form of a band on the leg of the clothing or in the form of a loop (3) which surrounds the base of the penis and can be detachably fixed to a second point on the clothing by a second fixing device (7) which is oriented away from the direction of the tractive force, on the shaft of the penis.

The present invention relates to a penis extension system that can be integrated into the clothing of a user and worn as the user moves around, with the aim of increasing the size of the penis and the glans. Said system comprises a cradle (1) which is used to receive the glans and can be fixed to the shaft of the penis. One end of a traction element (2) is fixed to the cradle and the other end of the traction element can be detachably fixed to a first point on the clothing (4) of a user by means of a first fixing device (7). The first point on the clothing depends on the tractive force and on the direction of the tractive force to be transferred to the penis by means of the tractive element (2). Said system also comprises an abutment arranged on the base of the penis of the user, said abutment being in the form of a band (4b) of the leg of the clothing or in the form of a loop (3) which surrounds the base of the penis and can be detachably fixed to a second point on the clothing (4) by a second fixing device (7) which is oriented away from the direction of the tractive force, on the shaft of the penis.

### BACKGROUND OF THE INVENTION

Devices for the permanent stretching of human body parts are known in various embodiments, and their effectiveness has been demonstrated in clinical tests.

Published devices for the stretching and permanent enlargement of the penis include for example:

### 1. WO1996026691 A1

### 2. WO2003073967 A1

### 3. DE19915407 A1

### 4. WO1999018897 A1

### 5. DE 10001331 A1

1. WO1996026691 A1 is a device whose extension element is formed by two rods that are mounted on a support ring that encircles the base of the penis, and through whose length adjustment an extension pressure is produced between the base of the penis and the glans, which is fixed at the other end of the rods by a fixing device. The device cannot be worn inconspicuously under clothing, and also entails health risks for the user. The length of the device during use results from the length of the flaccid penis to be stretched, which has an average length of 7-10 cm. Thus, when actually worn under the clothing, the plastic corner, protecting the right or left metal rod of the inherently rigid device, presses at a distance of 7-10 cm from the base of the penis against the important blood vessels that run there, such as the leg artery or pelvic artery, or the saphenofemoral junction. If the device is seated incorrectly, there is a risk that the support rods will press on the testicles, causing crushing of the testicles. The springs mounted in the support rods provide a yield of the tractive device of only about 5 mm. This is absolutely inadequate for pain-free sitting down and sudden movements of the user, because the penis, attached by tendons inside the body, requires play of at least two centimeters simply during the movement of sitting down.

For these reasons, this foreign element, not shaped to the anatomical features of the user's body, cannot be worn for long periods over the course of the entire day, assuming average activity on the part of the user. Therefore, it is doubtful that successful enlargement, which can be achieved only through long-lasting stretching, will be achieved using this device.

2. WO2003073967 A1 is constructed according to the same principle of operation as the above-described WO1996026691 A1, so that the criticisms of that device also pertain here. In addition, the stretching element (18 in the publication) has two solid tractive clips (18d in the publication) that press into the lower abdomen of the user when worn wider the clothing.

3. DE19915407 A1 stretches the penis in that the user guides the penis through the opening of a base plate and along a lifting column fixed to the base plate, and fixes the tip of the penis to a tractive fixing plane that can be displaced on the lifting column.

The use of this device is dangerous in that when the user makes a sudden movement, possibly injurious tractive forces act on the penis and could damage it, because the glans fixing plane is fixed to the lifting column by a screw. Also for this reason, it is not possible for the user to sit down or bend over while using the device. Because the penis is attached to the pelvic bone inside the body, a stretching device must be able to ensure some yielding. This device also cannot be worn inconspicuously under the clothing.

### 4. WO1999018897 A1

This is the only known device that can be worn inconspicuously under the clothing. A flexible, length-adjustable extension element, having a ring that acts as an abutment for the base of the penis, is wrapped around the user's body and is fixed at the other side to the user's glans. The resulting tractive forces cause a gradual enlargement of the penis.

A major disadvantage of the use of this device is the fastening of the extension element, which runs between the glans and the base of the penis, to the glans by means of a loop construction. The supply of blood to the glans is constricted in accordance with the tractive force, resulting in disturbances of the blood circulation, and, in the worst case, death of the glans. Moreover, the lever-type loop fixing presses the glans upward, opposite its normal position relative to the body of the penis, which, given long-term use, could result in changes to the penis and associated damage.

Putting on the device requires a high degree of effort, because the extension element that is to be wrapped around the body is constantly under tension due to its tractive property resulting from its design, so that the fixing of the glans requires a certain degree of skill.

### 5. DE 10001331 A1

This device, a further development of the above-discussed WO1999018897 A1, is identical in its stretching function, but as the fixing element for the glans it has, instead of the loop construction, a soft, small cap that can be pushed over the glans and that is intended to conform to the glans. The glans is to be held in this cap by a vacuum, and, as in WO1999018897 A1, connected to the extension element wrapped around the body. This fixing of the glans does not work. Because the glans does not have a secure hold, after a short time it withdraws out of this cap and is drawn far under the skin of the penis, so that only this skin is held by the vacuum and is drawn out and extended. Because of this, and through long maintenance of the vacuum, numbness of the glans may result, and the skin of the penis becomes slack and wrinkled; stretching of the penis itself does not take place at all.

All of the above-mentioned penis extension devices have the flaw that after only a short duration of stretching the glans becomes cold due to insufficient blood circulation and can take on a bluish coloring. The glans, which is essentially strangled in order to fix the distal end of the penis, does not develop in its size and circumference in a proportionate manner as a result of this treatment, and its growth lags behind. All of the devices are self-contained apparatuses having self-contained force circuits for installation on the naked male human body.

Their functionality is created when an object carrier on the glans is opposed to an abutment at the base of the penis, and a tractive force oriented in opposed directions is produced by some sort of extension element. Due to the fact that the traction and counter-traction by the connecting extension element operate at both ends of the penis, immediately and dependent on each other, there is the danger of a serious accident. In the worst case, the penis, which is anchored to the pelvic floor by tendons, could be torn out or seriously injured.

A schematic representation of the force circuits of the devices, compared alongside one another, discloses their identical functional principles, made up of three static elements, each serving the same purpose:
1. Fixing device for the glans
2. Connecting extension element
3. Abutment for the base of the penis.

The devices differ in their operation only by having different extension elements. WO1996026691 A1 and WO2003073967 A1 (FIG. 7, drawing S1) use two rods running in parallel as an extension element. DE19915407 A1 (FIG. 7, drawing S2) uses a lifting column as an extension element, while in WO1999018897 A1 and DE 10001331 A1 (FIG. 7, drawing S3) the extension element is realized as a band.

The setting of the tractive force acting on the penis takes place indirectly; it can be perceived and estimated only abstractly by a user not having knowledge of the device. Likewise, the donning of all these devices is very awkward and is possible only using both hands.

All of these applications are based on the idea of providing the user with a device in which he need only fix the penis, which is then stretched and extended. This abstract aim explains the awkwardness of these inventions, and does not take into account the fact that the penis is the most sensitive male body part.

The problem that the present invention endeavors to solve arises out of the above-described disadvantages of the known penis extension devices of this type.

### OBJECT OF THE INVENTION

The original, simplest form of penis extension is stretching it by hand. The user sees and feels the traction immediately, and intuitively pulls with the correct force.

Therefore, the aim of a stretching device must be to make use of this intuitive knowledge that each individual man has of his own loading capacity. Scales on the devices indicating newtons or pounds do not do justice to this individual sensitivity and therefore have only limited usefulness.

Therefore, the present invention is based on the object of stretching the penis directly and immediately with the hand, using the muscular force of the user's arm, and to hold it in this stretched state, with the ability to flexibly compensate possible counter-tractive forces due to movements such as sitting down or other bodily activities.

This bodily sequence of movements is now to be replaced by the present invention. Because no man would stretch his penis by pulling on the glans, the traction must be applied to the shaft of the penis and must protect the sensitive glans. Above all, the device must be capable of being used inconspicuously, without pain and without health risks, over an entire day during a standard day's work.

### TYPE OF APPLICATION

The present invention achieves this object through the features of patent claim 1, by means of a device in which, formulated graphically, the hand is replaced by a fixing element for the shaft of the penis, the arm is replaced by a tractive medium, and the shoulder is replaced by a fixing device. The body, as the base for the fixing, is replaced by the user's clothing, to which the tractive element and an abutment element can both be fixed independently of one another.

A schematic representation of the circuit of force (FIG. 7, drawings S4 and S5), illustrates the advantages, in comparison to the other applications shown there, of a fourth, external static element, the base element, which can be actively incorporated into the circuit of force and that makes it possible for the user to comfortably fix one or both independent tractive elements in a wide variety of directions with a wide variety of tractive forces and applications.
1a. Fixing device for the penis
2a. Separate tractive element for the penis
3a. Separate abutment for the base of the penis
4a. External base elements for fixing the tractive element and the abutment element.

The user puts on his underwear, which in this case represents the base element or clothing element. Subsequently, he fixes the penis in the glans cradle, which is connected not to the base element but rather to the tractive element. Next he decides in which direction he wishes to position the penis while it is being stretched, and pulls the tractive element in the desired direction. When the user has reached a tractive strength that is comfortable for him, he fixes the tractive element to a point in the underwear or clothing, using the fixing device. The fixing device is cushioned on its side facing the user during use, in order to prevent punctiform pressure on the skin.

Furthermore, if needed the user can position the abutment element, which is not connected to the tractive element, around the base of the penis or around the base of the penis and scrotum together, and can tension and fix it at a corresponding point on the underwear or clothing in the tractive direction opposite the tractive direction at the tip of the penis, using the fixing device of the abutment element. This provides an abutment against the tractive force acting at the tip of the penis, so that intensive traction acts on the body of the penis.

The user then has only to put on his outer trousers, which may even be tight jeans. It is now possible for him to wear this device inconspicuously for the entire day and to move about in unhindered fashion.

The present invention meets the requirements defined in the statement of the object, and represents significant progress in relation to the existing devices. The present invention is not an abstractly constructed additional foreign element, but, realized as underwear, fits the anatomical characteristics and standard clothing of men in a manner similar to the brassiere, which was invented one hundred years ago and which likewise performs shaping tasks for women.

Two independent tractive elements can be fixed to the clothing at any point as desired by the user, and can pull or provide tension in a large number of directions. Because there is no direct connection and transmission of force between the tractive element and the abutment element, accidents resulting from arbitrary, sudden actions of force are excluded.

In contrast to the above-mentioned closed systems, the present invention is an open system that uses external fixing possibilities, such as the clothing, as the fixing base. As such, the present device is novel in this market segment, and is distinguished from conventional, self-contained devices by its manner of functioning.

For the better understanding of the present invention, imagine a standing ladder that represents a self-contained, closed, non-integrated system for overcoming differences in height (FIG. 7, drawing S6). The present, open device is comparable to a leaning ladder, which is integrated into the existing external conditions and uses these conditions as a fixing base for overcoming a difference in height. It is easy to carry and can be used flexibly even given a very small ground surface (FIG. 7, drawing S6 [sic]).

The user is able to sit or to bend during use. The penis, connected to the pelvic bone inside the body by tendons, is not exposed to the danger of a tear of a tendon or tissue, because the flexible tractive element, in connection with the clothing, which is also flexible due to its material composition, provides sufficient play for a yield of at least 6 cm, despite the firmly set traction on the body of the penis.

The integration of a penis extension system into the standard clothing of the user greatly multiplies the static holding points, in comparison with all other self-contained extension devices, because both legs and the lower body of a user form the most authentic, because anatomically provided, foundation for this purpose.

Another advantage of the present invention is that the device can be tensioned and fixed, and also released, using only one hand.

Using the toilet is also problem-free. The user uses one hand to open the Velcro-type closure on the glans cradle, and can go about his business. The tractive element and abutment element remain connected to the trousers and cannot fall down.

In addition to the above-described advantages in comparison with the prior art, the penis extension system according to the present invention is also distinguished by its particularly protective fixing of the penis. The semicircular surrounding of the penis with the glans cradle, in the shape of a semicylindrical jacket, and the wide-surface sealing belt apply the force necessary for fixing not in a strangulating manner, but rather distributed over a large surface of the front part of the penis shaft, thus promoting blood circulation to the glans according to the principle of so-called erection rings and preventing reduced blood flow to the glans. In particular, sitting down and unhindered sport and professional activity are hereby possible without pain during use of the device.

The particular taking into account of a protective, blood circulation-promoting treatment of the glans by the vacuum container that is alternatively used as a fixing element is also new, and offers for the first time an intensive stretching of the body of the penis with simultaneous intensive circumferential volume expansion of the penis and glans, in order to ensure their proportionate growth relative to one another. This application is also to be worn under the clothing, but it is bulky enough that its wearing should be limited to the home.

Actual enlargement of the penis and glans can be achieved only by daily extension treatment for as long as possible. Due to its small components, flexible relative to one another and integrated independently into the clothing, the present device makes such long-term use possible over the whole day, during standard bodily activity in public, while remaining inconspicuous. In addition, the use of the present device can be expanded by the use in the home of the supplementary matching components such as the vacuum cylinder and weight training.

### PREFERRED EMBODIMENTS

A first preferred development of the penis extension device according to the present invention is as follows. A user places his penis into the glans cradle, which has the shape of a semicylindrical jacket and is connected to the tractive element, along its longitudinal axis, and fixes it in this cradle, behind the glans, by pulling a Velcro-type closure band which is fixed to the cylindrical jacket-shaped glans cradle transverse to the longitudinal axis thereof, following the radius of the cradle, over the shaft of the penis and the opposite longitudinal side of the glans cradle, and fixing it on the outer side, facing away from the penis, of the cylindrical jacket-shaped glans cradle by pressing against the fleece side of the Velcro-type band situated there. Next the user takes the other end of the tractive medium, on which is situated the fixing device for the clothing, in this case a suspender clip that is cushioned on its lower side facing the body of the user, and fixes this device to his underwear at a corresponding point, or to some other point of his clothing that seems to him to be appropriate, with a tractive strength that is comfortable for him.

It is then possible for him to wear the device inconspicuously and to move about in unhindered fashion for the entire day.

In order to exert a still more intensive tractive force on the body of the penis, the user can tension a separate abutment not connected to the tractive device, in the form of a cushioned, extensible loop that is placed around the base of the penis or the base of the penis and the scrotum together, in the tractive direction opposite the tractive direction at the tip of the penis, and can fix it to the fixing device for the abutment, in this case a tissue-protecting suspender clip cushioned on its underside, on his underwear or trousers, thus exposing the body of the penis to a sustained but yielding and flexible tractive tension.

Another preferred development of the penis extension device according to the present invention consists in that the tractive element is elastically expandable. The expandable traction improves wearing comfort and freedom of movement for the user, thus ensuring permanent stretching of the penis.

Another preferred development of the device consists in that the tractive element, connected to the fixing element for the penis, is exchangeable. Thus, the user has the possibility of using rubber bands having different tractive strength and elasticity, or non-expandable media, or to use a tractive element that is additionally adjustable in its length.

Another preferred development of the present invention consists in that the fixing device for fixing the tractive element to the clothing can also be fashioned as a Velcro-type closure whose counterpiece is fixed to the underwear of a user, so that the user can fix the tractive medium by pressing. A snap button closure, often used in textiles, or a simple standard button closure may also be used here. Also conceivable is the fixing of the tractive medium using a hook or eye closure, such as those often used in brassieres.

Another, more expensive variant provides the use of a rapid or clip closure having a counterpiece that can be detached by pressing it together using two fingers. Also conceivable is the use of a closure similar to a reusable cable zip tie connector, which has the advantage of limited length adjustability, or of a clamp or clasp closure, similar to a metal clamp, but in this case made of plastic, or the use of a closure similar to a very tightly closing clothespin.

Another preferred development of the penis extension system provides that the glans cradle is provided on its underside directly with a Velcro-type or other closure so that the glans cradle can be fixed with the desired tractive force directly at a corresponding point of the underwear, equipped in this case with the closure counterpiece, be it the fleece band of a Velcro-type closure or the counterpiece of a snap button.

Another preferred development of the present invention is distinguished in that the fixing element for fixing the tip of the penis is fashioned in the form of a semicylindrical jacket in such a way that it is open at its end face facing the user during use, so that a user can place his penis into the inner surface of the fixing element along the longitudinal axis in order to then fix it, behind the glans, using the cushioned band or belt closure that runs transverse to the longitudinal axis and that surrounds the fixing element and the penis laid therein. During use, the closed outer surface of the semicylinder faces the user, and is situated horizontally, approximately at the height of the groin. The outside of the cylinder, facing the user and curved in the manner of a semicircle, is fashioned so as to be smooth and able to slide, and enables the user to sit down without having to fear hindrance from sharp edges or corners, or injury to the groin.

Another preferred development of the present invention is distinguished in that the cushioned loop, which is not connected to the tractive device and which acts as an abutment against the tractive force on the tip of the penis, is extensible. As is the case with the fixing device for the tractive medium, there are many choices here for the fixing device, and the selection is primarily to be determined by the customer's wishes. However, here as well the tissue-protective suspender clip or clamp closure, cushioned on its side facing the user, is to be emphasized; it offers the advantage that it can be fixed at any point on the clothing independently and without having to attach a counterpiece.

Another preferred development of the penis extension system according to the present invention consists in that the clothing that acts as the fixing base for the tractive element and the abutment is fashioned as underwear in such a way that it has, around the front center part also known as the slot or access, where the penis and scrotum are located when the underwear is worn, fixing possibilities for the tractive element and the abutment element that are situated and fixed, or fixable, at a different distance, in order to enable the user to stretch the penis in various directions with different tractive forces and using different fixing devices.

Another variant embodiment of the penis extension system consists in that the fixing points, attached to the trousers acting as a base element, for the tractive element or for the abutment can also be used for other applications for shaping the sex organs. Conceivable here is a push-up function, as in a brassiere, in which a cushion is tensioned under the penis and the scrotum using the fixing points situated at both sides, causing the penis and scrotum to be lifted under the outer trousers, creating, for an observer not “in the know,” the impression of a larger volume of the sex organs.

Another preferred variant of the penis extension system consists in that the fixing points, attached to the trousers acting as base element, for the tractive element or for the abutment can also be used for other applications preparatory to sexual intercourse, in that the penis, or the penis and scrotum, are placed at their base between two abutment loops or holding belts pulling in the opposite direction. This measure prevents venous blood from flowing back out of the erectile tissue, resulting in a longer-lasting and stronger erection.

Another preferred development of the penis extension system according to the present invention consists in that the user has the possibility of selecting, instead of underwear as a base or fixing element, a belt system that surrounds the two legs individually with belts which are connected to one another by a web, forming, put graphically, a figure-eight. The belts are adjustable in their length and/or are made of an extensible material so as to be adaptable to the circumference of the thigh of the user. The user climbs into these two circular belts as into trousers, and pulls them up over the legs, into the crotch. It is then possible for the user to stretch his penis in the direction of the left or right hip joint, by first slipping the abutment of the leg belt facing away from the desired stretch direction of the penis over the penis or over the penis and the scrotum. He then fixes the glans in the glans cradle, which is connected not to the base element but rather to the tractive element. He then takes the free end of the tractive element and fixes it, with a tractive strength that he finds comfortable, to the corresponding point of the other leg belt, using the fixing device. Because the two belts encircling the legs are connected by a web, slippage due to the resulting traction is prevented.

Another preferred development of the penis extension system according to the present invention is distinguished in that the fixing element for the penis is made up of a plurality of media. A flat loop, connected at its underside to the run of the inner radius of the glans cradle, is used to receive the optional medium exerting the original binding pressure. This loop can be an extensible cable tie, a narrow leather or plastic arm band, a Velcro-type band, or an arbitrary rapid closure, because the skin does not come into contact with this element. On its inner side, and laterally, the flat loop is coated with a soft cushioned profile. The inventor has discovered that this type of “not immediate fixing” of the glans according to the present invention ensures good blood circulation to the glans, and prevents the glans from becoming cold or blue, in contrast to the prior art, even after being worn for a long period of time. The inventor is not a physician or medical professional and is therefore not able to say why this is so. Probably it is due to the fact that the originally constrictive medium can slide independently in the flat loop, which has completely different hardness and friction properties than does the soft coating material and the inner fixing medium.

Another preferred development of the present invention is distinguished in that the fixing element can be adjusted flexibly to the individual circumference of the penis that is to be fixed.

This is achieved in that the flat loop, fixed in the inner radius of the glans cradle, is open on its outer side, facing away from the glans cradle, for the closure of the binding medium. The inner, soft-coated side of the loop slides, as a tapering protective tongue, into the ring closure that now forms, thus ensuring a flexible inner radius.

Another preferred development of the system is distinguished in that the user of the penis extension system may also use, instead of the glans cradle as a fixing medium in connection with the tractive element, a vacuum container as a fixing medium in connection with the tractive element, in order to inventively carry out, simultaneous with the penis stretching, a strong expansion of the glans by a vacuum. However, this should be limited to thirty minutes per application in order to then ensure the return of normal blood circulation to the glans and to exclude the risk of otherwise possible tissue damage. Using an accompanying ball pump, the user pumps air out of the vacuum cylinder through the valve situated on tie side of the cylinder. The artificially produced vacuum causes more blood to flow into the glans, which as a result becomes enormously enlarged and expanded. This temporarily increased blood flow and expansion is intended to compensate the incorrect ratio between the body of the penis and the glans in this regard, and to promote growth of the glans in particular. As far as is currently known, the present invention is the only device that simultaneously enables the mechanical stretching of the penis and the large expansion and blood circulation to the glans produced by the vacuum. Behind the glans, facing the body of the user, the vacuum cylinder has a narrowing whose inner radius is adapted to the anatomical properties of the user, through which the user introduces his penis, and which offers a reliable holding of the penis against the tractive forces of the tractive element fixed to the tip. In order to enable the vacuum cylinder to be connected to the tractive element for penis stretching, it has at its tip on the outside a fixing device for the tractive element.

Another preferred development of the present invention is characterized in that the vacuum cylinder can be lengthened using an arrestable lengthening piece that fits circumferentially, in the manner of a telescope, over the part of the vacuum cylinder that faces the user's body during use. [and which] has, on its end not pushed over the vacuum cylinder and that lies on the user's body during use, a soft connecting sleeve that fits into the lengthening piece for support on the body of a user, through which the user introduces his penis. The user can determine the desired overall length of the cylinders pushed over one another by positioning the arresting clip, and thus has the possibility of exposing the penis to a short-term, very intensive stretching via vacuum, or of checking the success of previous stretching through a precise measurement of the length of the penis under vacuum.

Another preferred development of the penis extension system is distinguished in that a user who is sensitive to touch in the area of the groin can use, instead of the clothing or underwear as a base or fixing element for the tractive element for the penis and for the abutment element for the base of the penis, a corpus that is curved in the shape of a semicylindrical jacket and whose length is matched to the anatomical properties of the user and that reliably bridges over the area of the groin, with the blood vessels that run there such as the leg and pelvic artery and the saphenofemoral junction between the base of the penis and the hipbone or thigh, thus excluding any damage to these blood vessels. On an end surface facing the user, a base ring is fixed through which the penis of the user can be introduced, the glans cradle being situated inside the corpus so as to be able to slide along the longitudinal axis thereof, the glans of the penis of the user being, fixable to said cradle along its longitudinal axis, and the glans cradle being capable of being loaded via at least one tractive element that goes out from the glans cradle, the glans cradle being capable of being pivoted on the outer surface of the corpus over the end surface of the corpus that faces away from the user during use, and being capable of being fixed at a point on the outer surface of the corpus as a function of the tractive force that is to be set.

### DESCRIPTION OF THE DRAWINGS

In the following, the present invention is comprehensively explained in more detail with reference to FIGS. 1-25, which show preferred embodiments and various possible uses of the invention. The illustrations are not drawn to scale.

FIG. 1 is a view of the simplest application of the device in the tensioned state, in which for the sake of clarity fixing element (1), the glans cradle, is not shown in detail.

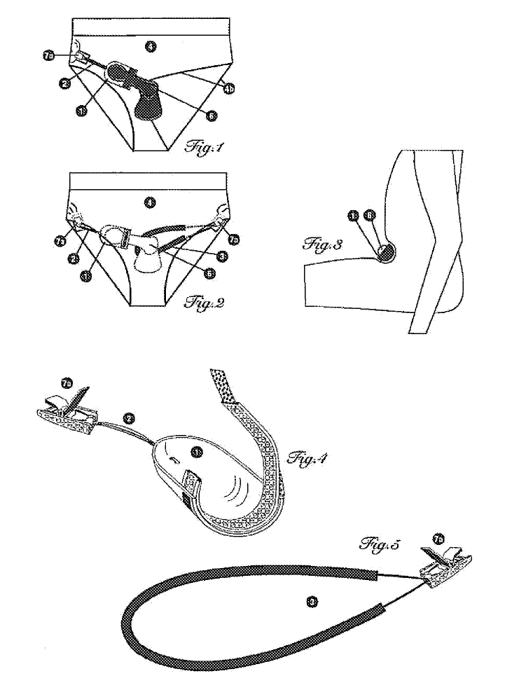

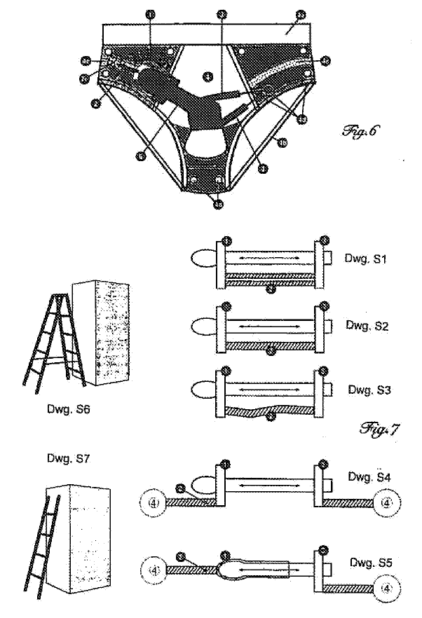

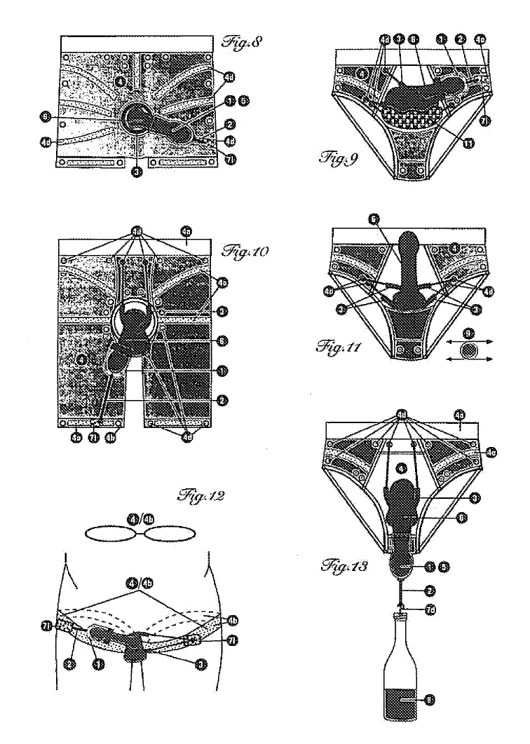

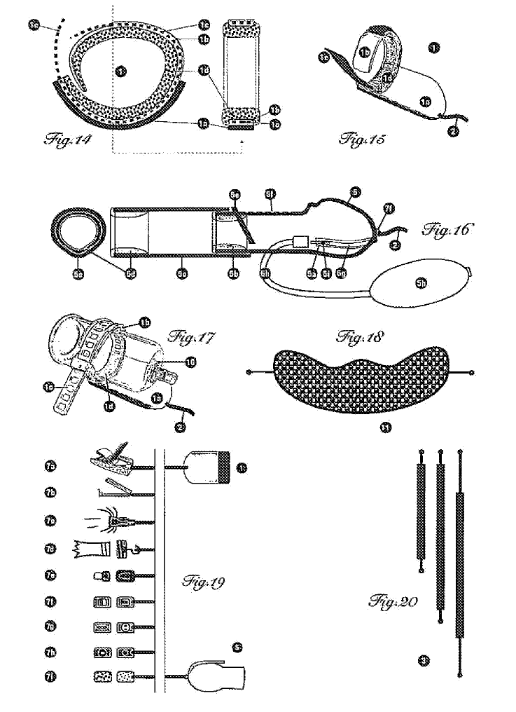

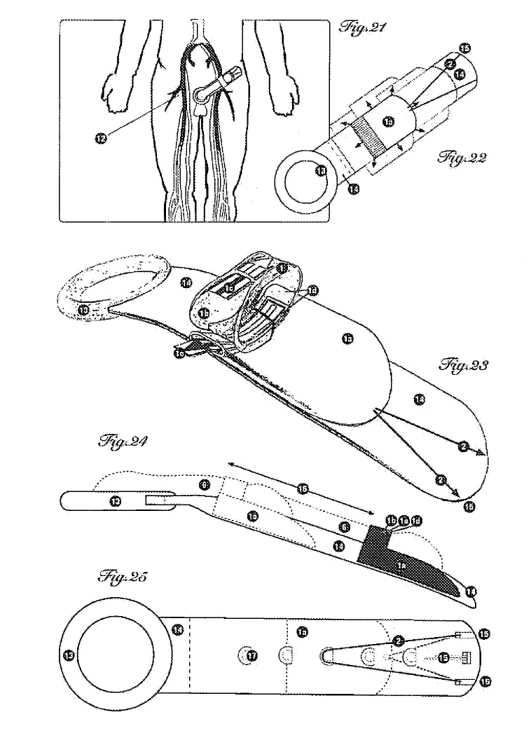

The user puts on underwear, which represents the base element, and slips leg band (4b) situated opposite the desired direction of traction of the penis over the penis and scrotum in such a way that these are situated outside the underwear. Subsequently, the user fixes penis (6) to glans cradle (1) along its longitudinal axis; the cradle is not yet connected to base element (4), in this case the underwear, but rather to extensible tractive element (2). The user then pulls on the free end of extensible tractive element (2), which in this case has a suspender clip (7a) as fixing device, and feels the resulting tractive forces on the body of the penis. He then fixes suspender clip (7a), which is connected to extensible tractive element (2), to a corresponding point on underwear (4) with a tractive force that he finds comfortable. The tractive device now exerts a traction on the body of the penis (6) that is permanent but that is adapted to the user's movements due to the high flexibility.

FIG. 2 shows the use of the device, worn under conventional underwear, in the tensioned state with the use of abutment element (3). The user first passes abutment loop (3) over penis (6), or over penis (6) and scrotum together, and fixes suspender clip (7a), used here as an example as fixing device and cushioned on its underside, to leg band (4b) of the underwear from inside. Using belt (1c), the user subsequently fixes penis (6) to glans cradle (1a) along its longitudinal axis, said cradle being connected to tractive element (2), and pulls on the other side of the cradle, where a suspender clip (7a) is situated as a fixing device (7), and fixes this clip, with the desired tractive force on the body of the penis, from the inside to the other leg band (4b) situated opposite the direction of traction of abutment element (3). The device is now barely perceptible by a third party, even if the user is wearing only underwear or gym shorts.

FIG. 3 shows the position of glans cradle (1) during sitting. Due to the smooth, round shape of the glans cradle, a punctiform pressure is avoided, in contrast to the prior art. The user can sit down and bend in unhindered fashion with no health risks.

FIGS. 4 and 5 show the basic equipment of the device, intended for use with commercially available clothing or underwear, consisting of fixing element (1) for the penis, with tractive element (2) and fixing device (7a), as well as abutment element (3) with fixing device (7a).

FIG. 6 shows the device in the tensioned state with the use of a functional undergarment (4) constructed for the penis extension system. The part known as the access, in the front center of the underwear, where penis (6) and the scrotum of a user are situated, is left open so that the underwear are outwardly open and accessible in this area but can be covered with the access part using snap buttons (4c and d). At both sides, as well as above and below this area, the underwear has fixing devices (4c and d), here in the form of counter pieces to snap buttons as well as a Velcro-type band (7i) as a counter piece to fleece belt (7i). The user fixes fixing device (7i) of the extensible tractive element (2), which in this case is equipped with the hook side of a Velcro-type closure (7i) to the point of the fleece belt attached to the underwear corresponding to the desired traction. The user does the same with abutment element (3). In order to maintain a firm hold on the scrotum, this underwear is equipped with a suspensory device in order to accommodate the scrotum.

FIG. 7 shows, in drawings 1 to 3, the diagram of forces of the devices compared alongside one another, and, in drawings 4 and 5, the differing diagram of forces of the present invention; drawings 6 and 7 show examples of a standing ladder and a leaning ladder.

FIG. 8 shows an undergarment in short form in which abutment (3), acting on all sides, for penis (6) is formed by an embedded ring. Velcro-type bands (7i), used as examples, which run in the shape of a star from the direction of the base of the penis, enable tractive element (2) to be fixed in various directions. Here, as an example vacuum container (5) is used instead of the glans cradle as the fixing element for the penis connected to extensible tractive element (2).

FIG. 9 illustrates the use of a push-up cushion (11). The user fixes a cushion underneath the penis and the scrotum by means of fixing points (4c and d) situated at both sides of the penis and scrotum on base element (4). In addition to this, the user can now also carry out a stretching, but because of the cushion, which presses upward, this is limited to stretching in the direction of the right or left hip. In this view, the user carries out a stretching in the direction toward the left hip, as seen by the user.

FIG. 10 shows the stretching of penis (6) using a knee-length garment as base element (4). Abutment (3) is held in the center underneath pants band (4a) by two holding points (4d), and thus exerts the counterpressure to extensible tractive element (2), which is fixed, running downward at a steep angle in the direction of the knee, to right leg band (4b), as seen by the user.

FIG. 11 shows the use of base element (4) with two abutments (3) as an aid against what is known as erectile dysfunction, in that penis (6), or the penis and the scrotum, are placed at their base between two abutments (3) pulling in the opposite direction, fixed to two respective holding points (4d) of base element (4). By means of this measure, the back-flow of the venous blood out of the erectile tissue of penis (6) is prevented, and a longer-lasting, stronger erection results.

FIG. 12 shows the use of alternative base part (4/4b), encircling only the legs in the shape of a figure eight, using the glans cradle as fixing element (1) for the penis. As an example, a Velcro-type band is used for the continuously adjustable fixing of tractive element (2) and of abutment element (3). Because the two leg belts are connected by a web in the crotch, they are prevented from slipping relative to each other.

FIG. 13 is based on the origin of penis stretching as practiced by traditional cultures. However, the stretching practiced by these people did not make use of an abutment (3). Abutment (3) is held by two holding points (4d) on base element (4), in the center underneath the trouser band (4a), and thus exerts the counterpressure to extensible tractive element (2), which runs steeply downward, as seen by the user, and is fixed to a weight (8) between the user's legs. In this case, the fixing element for the penis is vacuum cylinder (5). Weight (8) is for example a bottle filled with sand on whose screw closure tractive element (2) is fixed using a hook (7d) as fixing device (7).

FIG. 14 shows fixing element (1) in section and from the side, said fixing element being fixed in circular fashion in the inner radius of glans cradle (1a) to flat loop (1b) that receives binding medium (1c). On its inside, the flat loop is coated with a cushioned profile (1d). In the area of the circular form situated opposite the inner radius of the glans cradle, the flat loop opens outwardly in order to permit the exit of binding medium (1c). The inside, coated with cushioned profile (1d), of flat loop (1b) is pushed over the other end of flat loop (1b) and in this way inwardly closes the circular fixing element. The end of binding medium (1c) that exits externally from flat loop (1b), which is attached in circular fashion to the inner radius of the glans cradle, is now connected to its other end and is closed with a binding pressure that is felt to be comfortable by the user. As a binding medium, a wide variety of closures are possible, because they do not come into contact with the skin.

FIG. 15 shows fixing element (1) made up of glans cradle (1a) with flat loop (1b) and a Velcro-type closure as a binding medium (1c), shown as an example and arranged in the loop.

FIG. 16 shows a section of cylindrically shaped vacuum container (5) according to the present invention for expanding the glans with simultaneous stretching of the penis through connection to tractive element (2), which is connected to base element (4) or to the base element corpus (14), or to a weight (8), or to lengthening part (5c). The front part, for receiving the glans, is shaped corresponding to the anatomical properties of the user, while the rear part, through which the user introduces his penis, has a removable sleeve (5b) so that it can accommodate different penis sizes. The user guides the glans in front of sleeve (5b) of vacuum container (5). Using the accompanying vacuum pump, the user then pumps the air out of vacuum container (5) via the cylindrical air duct, which runs parallel to the glans form and is open towards the interior along its longitudinal axis, and that has at its end facing the user during use a connecting part (5a) having a valve. This has the advantage that punctiform, injurious suctioning of a point on the skin can be excluded. Due to the resulting vacuum, the glans is drawn firmly into the vacuum container and expands enormously due to the blood flowing in, until it completely fills the container. The removable narrowing (5b) ensures that the penis is firmly fixed in the container and cannot slip back. The user then removes vacuum pump (5h) and fixes the vacuum container to base element (4) in the same manner as glans cradle (1a), in order to achieve a stretching of the body of the penis at the same time as the expansion of the glans. That is, he pulls on tractive element (2), which is attached to the tip of vacuum container (5) by an eye fastener (7D), and fixes this tractive element, using suspender clip (7a) as fixing device, which is fixed at the other end of the tractive element, to the location on base element (4) or (14) corresponding to the desired traction or to weight (8). In order to terminate this use after approximately thirty minutes, in order to release the vacuum he actuates the button on vacuum valve (5i) in order to withdraw the penis from the container. For stretching without a base element, or in order to measure the length of the penis in the erect state, the user lengthens vacuum container (5) by sliding the likewise cylindrical lengthening part (5c) over vacuum container (5), whose shape is identical in the rear area up to the beginning of the glans formation but whose diameter is smaller, and snaps in arresting clip (5e) at the desired length, at the corresponding catch (5f) on vacuum container (5).

FIG. 17 shows a perspective view of a readily commercially available conventional vacuum container (5), fixed to fixing element (1) by means of extensible cable ties (1c), used here as an example, on glans cradle (1a). This container has the disadvantage that the air connecting part in the front part can result in pressure points and injury to the glans of the user when the vacuum is produced.

FIG. 18 shows the push-up cushion (11) for lifting the penis and scrotum, to be connected at both sides to base element (4) using fixing points (4d).

FIG. 19 shows a selection of preferred possible connections (7a-7i) between base element (4) and tractive element (2), which is connected at its other side optionally to glans cradle (1a) or to vacuum container (5) for receiving the tip of the penis.

FIG. 20 shows cushioned abutment elements, having different lengths, that are to be fixed to base element (4) and are to be used as abutments (3) at the base of the penis.

FIG. 21 shows the position of blood vessels (12) in the groin. The design according to the present invention of the corpus base element (14) bridges over this area due to its length, which is matched to the anatomical characteristics of the user; during use its closed longitudinal side, having the shape of a cylindrical jacket, faces the body of the user, so that the user is protected from sharp corners and edges or rigid parts. In contrast, all known penis extension devices of this type have dangerous parts in this area.

FIG. 22 shows glans cradle (1a), which is capable of sliding in the semicylindrical jacket-shaped base element corpus (14) and is situated along the longitudinal axis thereof and is movable towards all sides, said cradle being drawn, pivotably via the guide of corpus tip (15), by tractive element (2) for fixing on the outer side thereof.

FIG. 23 is a perspective overall view of the base element corpus with glans cradle, in which for the sake of clarity the cushion profile (1d, FIG. 14) of fixing element (1) is not shown.

The user guides the penis through base ring (13), which is connected fixedly to corpus (14), and now has both hands free. He then takes in one hand a glans cradle (1), which is not yet connected to the corpus, and with the other hand he places his penis in the glans cradle along its longitudinal axis, in such a way that the tip of the glans points toward the fixing device of the tractive element attached to the rounded end face of the glans cradle. The circular belt closure of glans cradle (1b-1d) now encircles the shaft of the penis behind the glans, and is closed using binding element (1c). With one hand, the user now moves base ring (13) towards the base of the penis and holds it firmly there. With the other hand, the user grasps tractive element (2) on which the tip of the penis, fixed to the glans cradle by fixing element (1), is situated, and pulls this tip, in the guide of cylindrical jacket-shaped corpus (14), in the direction of corpus tip (15). The user feels the resulting tractive forces on the body of the penis. He guides tractive element (2) using guide (15) on the tip of corpus (14), and fixes tractive element (2) with a tractive tension that he finds comfortable at a fixing point (17) corresponding to this tension, countersunk in the rear side of the corpus. The self-contained force system now exerts a traction on the body of the penis that is permanent but that is adapted to the movements of the user due to its high flexibility.

FIG. 24 is a side view of the penis extension device, or a top view of the user during use, showing the position of penis (6) and of the glans. In this view, the function of corpus (14) for bridging over the area of the large blood vessels that run here can be assessed particularly well.

The arrow (16) at both sides represents the range of traction to which the body of the penis is exposed.

FIG. 25 shows the closed outer side, facing the user during use, of the semicylindrical jacket-shaped base element corpus (14) at whose countersunk fixing points (17) the tractive elements pivotable via the tractive element guide (15) on the tip of the corpus, is tensioned and fixed.

### LIST OF REFERENCE CHARACTERS

1 fixing element
   1a glans cradle
   1b flat loop
   1c binding medium
   1d cushioned profile
2 tractive element
   2a length-adjustable tractive element
3 abutment for the base of the penis
4 base element
   4a trouser band
   4b leg band
   4c fixing devices for tractive element
   4d fixing devices for abutment
5 vacuum container
   5a connecting part
   5b sleeve of vacuum container
   5c lengthening part
   5d sleeve of lengthening part
   5e arresting clip
   5f arrest points
   5g laterally open air duct
   5h pump
   5i valve Reference characters for FIG. 7: 6 penis 1 fixing device for the glans 7 fixing device 2 connecting expansion element 7a suspender clip 3 abutment for the base of the penis 7b clamp closure 7c clasp closure 1a fixing device for the penis 7d hook for screw closure 2a separate tractive element for the penis 7e quick-acting closure 3a separate abutment for the base of the penis 7f eye closure 4a external base element for fixing the tractive 7g button closure element and the abutment element 7h snap button closure 7i Velcro-type closure
8 weight
9 traction diagram
10 external vacuum container
11 push-up cushion
12 arteries/veins
13 abutment/base ring
14 base element corpus
15 tractive element guide
16 traction range
17 countersunk fixing device

1. A penis extension system that can be integrated into the clothing of a user and worn as the user moves around, for increasing the size of the penis and the glans, having a cradle (1) which is used to receive the glans and can be fixed to the shaft of the penis, to which cradle one end of a traction element (2) is fixed while the other end of the traction element can be detachably fixed to a first point on the clothing (4) of a user by means of a first fixing device (7), the first point on the clothing depending on the tractive force and on the direction of the tractive force to be transferred to the penis by means of the tractive element (2), said system also comprising an abutment arranged on the base of the penis of the user, said abutment being in the form of a band (4b) of the leg of the clothing or in the form of a loop (3) which surrounds the base of the penis and can be detachably fixed to a second point on the clothing (4) by a second fixing device (7) which is oriented opposite to the direction of the tractive force on the shaft of the penis.

2. The penis extension system as claimed in claim 1,
wherein the article of clothing (4) is a set of pants (4).

3. The penis extension system as claimed in claim 1.
wherein the article of clothing (4) is formed by at least one band (4b) that encircles both thighs of a user, resulting in a belt system in the shape of a figure eight.

4. The penis extension system as claimed in claim 3,
wherein the at least one band (4b) is length-adjustable and/or extensible.

5. The penis extension system as claimed in claim 1,
wherein the tractive element (2) is elastically extensible.

6. The penis extension system as claimed in claim 1,
wherein the tractive element (2) is length-adjustable.

7. The integrated penis extension system as claimed in one or more of the preceding claims,
wherein the semicylindrical jacket-shaped fixing element (1a) for the penis partially surrounds the front area of the penis in its circumference, radially along the longitudinal axis, and partially surrounds the tip of the penis transverse to the longitudinal axis of the penis.

8. The integrated penis extension system as claimed in one or more of the preceding claims,
wherein the forces engaging the penis of a user via the tractive element (2) and via the abutment (3) are directed opposite one another.

9. The integrated penis extension system as claimed in one or more of the preceding claims,
wherein the first and/or the second fixing device (7) is fashioned as a material clip, known as a suspender clip (7i).

10. The integrated penis extension system as claimed in one or more of the preceding claims,
wherein the penis of a user can be fixed by at least one belt-type closure (1b-1d) fixed to the glans cradle (1a).

11. The integrated penis extension system as claimed in one or more of the preceding claims,
wherein the glans cradle (1a) is fashioned as a vacuum container (5).

12. The integrated penis extension system as claimed in claim 11,
wherein the vacuum container (5) has, along the front part for receiving the glans, a cylindrical duct (5g) that runs parallel to the interior of the vacuum container (5) and is open thereto along its longitudinal axis, and that is provided with a connecting part (5a) on its open end face facing a user during use.

13. The integrated penis extension system as claimed in claim 11,
wherein the vacuum container (5) has in its rear part facing the body, through which a user introduces his penis, a removable circular narrowing or sleeve (5b) that fits into the opening of the vacuum container.

14. The integrated penis extension system as claimed in claim 11,
wherein the vacuum container (5) has a lengthening part (5c) that extends over the shaft of the penis of a user during use, and that can be fixed on the vacuum container (S) at an arrest (5f), and that has at its opening facing the body during use a removable circular narrowing or sleeve (5d) that fits into the opening of the lengthening part (5c).

15. A penis extension system to be worn as the user moves around, for increasing the size of the penis and the glans, having an open corpus (14) that is curved in the shape of a semicylindrical jacket, on whose end face facing a user during use there is fixed a base ring (13) through which the penis (6) of a user can be introduced, a glans cradle (1a) being arranged in the inner surface of the corpus (14) so as to be capable of sliding along its longitudinal axis, on which cradle the glans of the penis of a user can be fixed along its longitudinal axis, and the glans cradle being loadable by at least one tractive element (2) that goes out from the glans cradle (1a) and that is pivotable on the external surface of the corpus (14) over the end face facing away from the user during use of the corpus (14) and that can be fixed to a point (17) on the external surface of the corpus dependent on the tractive force that is to be set, in that the corpus (14) is longer than the maximum length of the penis of a user.

16. The penis extension system as claimed in claim 15,
wherein the semicylindrical jacket-shaped, curved external side of the corpus (14) faces the body of a user during use.
